(19) United States
(12) Patent Application Publication
(10) Pub. No.: US 2026/0097140 A1
(43) Pub. Date: Apr. 9, 2026

(54) Radiolabelled Compounds

(71) Applicants: THE UNIVERSITY OF MELBOURNE, Victoria (AU); PETER MACCALLUM CANCER INSTITUTE, Victoria (AU)

(72) Inventors: Asif NOOR, Victoria (AU); Peter Douglas ROSELT, Victoria (AU); Paul Stephen DONNELLY, Victoria (AU)

(21) Appl. No.: 19/114,340

(22) PCT Filed: Sep. 21, 2023
(86) PCT No.: PCT/AU2023/050914

§ 371 Date: Mar. 21, 2025

(30) Foreign Application Priority Data
Sep. 21, 2022 (AU) 2022902725

(51) Int. Cl. A61K 51/10; A61K 51/08; C01G 25/04

(52) U.S. Cl. CPC A61K 51/1075; A61K 51/083; A61K 51/088; A61K 51/1093; C01G 25/04; A61K 2121/00; C01P 2006/88

(57) ABSTRACT

Processes for the synthesis of [⁸⁹Zr]ZrCl₄ from [⁸⁹Zr][Zr(oxalate)₄]⁴⁻ salt are provided. The [⁸⁹Zr]ZrCl₄ can be reacted with biomarker targeting agents to produce ⁸⁹Zr labelled radiopharmaceuticals. The ⁸⁹Zr labelled radiopharmaceuticals find use in, for example, non-invasive molecular imaging.

### FIELD OF THE DISCLOSURE

The present disclosure relates to the synthesis of [⁸⁹Zr]ZrCl₄ from [⁸⁹Zr][Zr(oxalate)₄]⁴⁻ salt. The disclosure also relates to the synthesis of ⁸⁹Zr labelled radiopharmaceuticals from [⁸⁹Zr]ZrCl₄ and biomarker targeting agents. The ⁸⁹Zr labelled radiopharmaceuticals find use in, for example, non-invasive molecular imaging.

### BACKGROUND OF THE DISCLOSURE

Zirconium-89 is a positron-emitting radionuclide that is used for Positron Emission Tomography (PET). PET using antibodies for targeting has become an important molecular imaging technique in cancer diagnosis and therapy. Zirconium-89 (⁸⁹Zr) has emerged as one preferred radiometal for radiolabelling of antibodies because of its availability, cost, and relative ease of radiolabelling. The radionuclide has a radioactive half-life of 78 hours which means it has traditionally been used to radiolabel antibodies that require several days to clear from background and accumulate in target tissue. There has been recent interest in using zirconium-89 as a ‘centrally manufactured radiopharmaceutical’ where the relatively long radioactive half-life enables distribution and supply of pre-made radiopharmaceuticals to geographically distant locations.

Zirconium-89 is commercially available from several sites, including Perkin Elmer (produced in The Netherlands and distributed globally) as well as in Melbourne, Australia, by the Austin Hospital. The radionuclide is typically supplied as ‘zirconium-89 oxalate’. The very low concentration of radionuclide is typically provided in 1 M oxalic acid. The oxalic acid is essential for the purification and isolation of the manufactured radionuclide, but must be removed before the zirconium-89 is incorporated in bespoke biological targeting agents (for example, chelator-peptide or chelator-antibody constructs). The commercially provided mixture of zirconium-89 must be both neutralised (it is too acidic to form complexes with the chelator) as well as the toxic oxalic acid removed. This removal of oxalic acid and neutralisation step is traditionally performed by an on-site radiochemist who adds sodium carbonate and monitors pH. The process is tedious, time consuming and involves considerable manual handling of a radioactive isotope. This step also significantly dilutes the concentration of the zirconium-89 and can lead to losses of radioactivity due to colloid formation/physiochemical adsorption of radionuclide to the precipitated oxalate.

As an alternative to [⁸⁹Zr]Zr-oxalate, [⁸⁹Zr]ZrCl₄ has been examined as a zirconium precursor (Holland, J. P., et al, Nucl Med Biol. 2009 October; 36 (7): 729-739). The [⁸⁹Zr]ZrCl₄ was prepared by loading [⁸⁹Zr]Zr-oxalate onto a strong anion exchange cartridge in chloride form which was pre-activated with acetonitrile and then eluting with 1 M aqueous HCl. The [⁸⁹Zr]ZrCl₄ was then reconstituted in either water, saline or 0.1 M aqueous HCl for further use. However, the use of acetonitrile is not ideal for clinical applications, and the high concentration of HCl (1 M) used for elution requires further post-elution steps to reduce the HCl concentration.

In view of the foregoing there is an ongoing need to develop improved processes for the synthesis of zirconium-89 precursors which may be useful in the production of zirconium-89 radiopharmaceuticals.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE DISCLOSURE

The present disclosure describes new processes for the synthesis of [⁸⁹Zr]ZrCl₄ from [⁸⁹Zr][Zr(oxalate)₄]⁴⁻ salt. The processes advantageously produce solutions of [⁸⁹Zr]ZrCl₄ which may be used directly in the preparation of radiopharmaceuticals for imaging or therapeutic applications, obviating the need for lengthy purification or handling procedures. Additionally, the processes are amenable to automation, which reduces the risk of operator exposure to radiation.

In one aspect the present disclosure provides a process for the synthesis of [⁸⁹Zr]ZrCl₄ solution comprising:
(a) contacting a solution comprising [⁸⁹Zr][Zr(oxalate)₄]⁴⁻ salt with a porous solid having anion exchange capacity, said porous solid comprising ligands covalently attached thereto, said ligands comprising ion exchange groups having a positive charge;
(b) treating the porous solid with an acidic solution comprising chloride ions; and
(c) recovering a solution comprising [⁸⁹Zr]ZrCl₄ from the porous solid.

In embodiments, the porous solid having anion exchange capacity comprises hydrogen carbonate ions, carbonate ions, hydrogen phosphate ions, phosphate ions, chloride ions, or mixtures thereof.

In embodiments, the porous solid having anion exchange capacity comprises hydrogen carbonate ions, carbonate ions, or mixtures thereof.

In embodiments, the porous solid having anion exchange capacity comprises hydrogen carbonate ions.

In embodiments, the porous solid is in particulate form.

In embodiments, the porous solid is disposed in a packed bed.

In embodiments, the porous solid comprises synthetic organic polymer, silica or alumina.

In embodiments, the synthetic organic polymer comprises crosslinked polystyrene-divinylbenzene.

In embodiments, the ligands comprising ion exchange groups having a positive charge comprise quaternary ammonium groups, or quaternary phosphonium groups.

In embodiments, the acidic solution comprising chloride ions comprises HCl.

In embodiments, the concentration of HCl in the acidic solution comprising chloride ions is less than 1 M, or less than about 0.5 M, or less than about 0.2 M.

In embodiments, the concentration of HCl in the acidic solution comprising chloride ions is from about 0.01 M to less than 1 M, or from about 0.05 M to about 0.5 M, or from about 0.05 M to about 0.2 M.

In embodiments, the acidic solution comprising chloride ions further comprises alkali metal chloride, for example sodium chloride.

In embodiments, the concentration of alkali metal chloride is from about 0.1 M to about 2 M, or from about 0.5 M to about 1.5 M.

In some preferred embodiments, the acidic solution comprising chloride ions comprises HCl in a concentration from about 0.05 M to about 0.5 M, and alkali metal chloride in a concentration from about 0.5 M to about 1.5 M.

In some preferred embodiments, the acidic solution comprising chloride ions comprises HCl in a concentration from about 0.05 M to about 0.2 M, and alkali metal chloride in a concentration from about 0.5 M to about 1.5 M.

In embodiments, the solution comprising [⁸⁹Zr]ZrCl₄ has a pH greater than 1.

In embodiments, the solution comprising [⁸⁹Zr]ZrCl₄ has a pH greater than about 2, or greater than about 3, or greater than about 4, or greater than about 5, or greater than about 6.

In embodiments, the process is performed in an aqueous environment. In embodiments, the acidic solution comprising chloride ions is an aqueous solution comprising chloride ions.

In embodiments, the process is free of organic solvents.

In embodiments, the yield of [⁸⁹Zr]ZrCl₄ is at least about 80%, or at least about 85%, or at least about 90%, or at least about 95%, based on [⁸⁹Zr][Zr(oxalate)₄]⁴⁻ salt.

In embodiments, the porous solid having anion exchange capacity comprises hydrogen carbonate ions, the concentration of HCl in the acidic solution comprising chloride ions is less than 1 M, or less than about 0.5 M, or less than about 0.2 M, and the acidic solution comprising chloride ions further comprises alkali metal chloride, for example sodium chloride, and the concentration of alkali metal chloride is from about 0.1 M to about 2 M.

In another aspect the present disclosure provides a process for the synthesis of a ⁸⁹Zr labelled radiopharmaceutical comprising the step of contacting a solution of [⁸⁹Zr]ZrCl₄ formed by the process according to any one of the herein disclosed embodiments with a biomarker targeting agent, said biomarker targeting agent comprising one or more moieties capable of forming a complex with zirconium of coordination number six to eight.

In embodiments, the biomarker targeting agent comprises a small molecule, or a peptide.

In embodiments, the small molecule has a molecular weight of less than 1000 Dalton.

In embodiments, the biomarker targeting agent comprises one or more of polypeptide, protein, and antibody.

In embodiments, the one or more moieties capable of forming a complex with zirconium is a chelator.

In embodiments, the chelator comprises one or more nitrogen, oxygen, or sulphur atoms.

In embodiments, the chelator is selected from DFO-squaramide, DFO*-squaramide, benzyl isothiocyanate-DFO, benzyl isothiocyanate-DFO*, wherein DFO is desferrioxamine B and DFO* is desferrioxamine*, and DOTA.

In embodiments, the biomarker targeting agent is selected from DFOSq-bisPSMA, DFOSq-octreoTATE, DFOSq-girentuximab, and DOTA-octreotate.

In embodiments, any one or more of the herein disclosed process steps may be automated.

In another aspect the present disclosure provides a solution of [⁸⁹Zr]ZrCl₄ formed by a process according to any one of the herein disclosed embodiments.

In another aspect the present disclosure provides a ⁸⁹Zr labelled radiopharmaceutical formed by a process according to any one of the herein disclosed embodiments.

In another aspect the present disclosure provides a ⁸⁹Zr labelled radiopharmaceutical formed by a process according to any one of the herein disclosed embodiments for use in the treatment of cancer in a patient.

In another aspect the present disclosure provides a method of treating cancer in a patient, the method comprising administering to the patient a ⁸⁹Zr labelled radiopharmaceutical formed by the process according to any one of the herein disclosed embodiments.

In another aspect the present disclosure provides a ⁸⁹Zr labelled radiopharmaceutical formed by the process according to any one of the herein disclosed embodiments for use in targeting a biomarker in vivo.

In another aspect the present disclosure provides a method of targeting a biomarker in vivo, comprising administering to a subject a ⁸⁹Zr labelled radiopharmaceutical formed by the process according to any one of the herein disclosed embodiments.

Biomarkers include, but are not limited to, PSMA, bombesin, CAIX, FAP, and HER2.

Advantages of the presently disclosed processes may include one or more of the following:
solutions of [⁸⁹Zr]ZrCl₄ may be prepared free of organic solvents, particularly potentially toxic organic solvents;
the process effectively removes oxalic acid/oxalate;
due to the low acid concentrations, solutions of [⁸⁹Zr]ZrCl₄ may be directly utilised for the preparation of ⁸⁹Zr labelled radiopharmaceuticals;
reduced operator exposure to radiation.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and processes are clearly within the scope of the disclosure, as described herein.

Further aspects of the present disclosure and further embodiments of the aspects described in the preceding paragraphs will become apparent from the following description, given by way of example and with reference to the accompanying drawings.

### DETAILED DESCRIPTION OF THE EMBODIMENTS

It will be understood that the disclosure described and defined in this specification extends to all alternative combinations of two or more of the individual features mentioned or evident from the text or drawings. All of these different combinations constitute various alternative aspects of the disclosure.

### Definitions

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps.

“About” as used herein when referring to a measurable value such as an amount, a temporal duration, and the like, is meant to encompass variations of ±20% or ±10%, in some instances±5%, in some instances±1%, and in some instances±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, the term “radiopharmaceutical” refers to an agent that contains a radioactive substance which is used to diagnose or treat disease, for example cancer. Radiopharmaceuticals may be used in non-invasive molecular imaging or to deliver a therapeutic dose of ionising radiation to tissue.

As used herein, the term “biomarker targeting agent” refers to an agent which comprises both functionality capable of forming a complex with zirconium and functionality which targets one or more biomarkers in vivo.

As used herein, the term “ion-exchange group” refers to an ionic group or an ionizable group. Ionic groups are charged (e.g., positively charged quaternary amine), while ionizable groups can be charged or non-charged depending on the conditions to which the ionizable group is exposed (i.e., basic or acidic groups). For example, a tertiary amino group can be charged by accepting a proton (basic group). Anion-exchange groups include primary, secondary, tertiary and quaternary amines, as well as any other basic (proton-accepting) functionalities.

In one aspect the present disclosure provides a process for the synthesis of [⁸⁹Zr]ZrCl₄ solution comprising:
(a) contacting a solution comprising [⁸⁹Zr][Zr(oxalate)₄]⁴⁻ salt with a porous solid having anion exchange capacity, said porous solid comprising ligands covalently attached thereto, said ligands comprising ion exchange groups having a positive charge;
(b) treating the porous solid with an acidic solution comprising chloride ions; and
(c) recovering a solution comprising [⁸⁹Zr]ZrCl₄ from the porous solid.
   [⁸⁹Zr][Zr(Oxalate)₄]⁴⁻ Salt

In embodiments, the ⁸⁹Zr source utilised in the presently disclosed processes is aqueous zirconium oxalate. Oxalate is used to assist in the purification of zirconium (IV) and stabilize the ion in solution, and is the typical commercial source of ⁸⁹Zr, but this oxalate has to be removed prior to the preparation of radiopharmaceuticals.

The [⁸⁹Zr][Zr(oxalate)₄]⁴⁻ salt is typically provided as a solution in 0.05 to 1 M oxalic acid.

### Porous Solid

The porous solid of the present disclosure comprises a solid support comprising covalently attached ligands. The ligands comprise ion exchange groups having a positive charge.

The solid support of the present disclosure can be any solid material that is characterized by pores (e.g., those useful as a stationary phase/packing material for chromatography). In one example, the solid support includes inorganic (e.g., silica) material. In another example, the solid support includes organic (e.g., polymeric) material (e.g., synthetic resins). In yet another example, the solid support includes a hybrid inorganic-organic material. The solid support is preferably insoluble in the solvent system used for a particular separation.

In one embodiment, the solid support includes metal oxides or metalloid oxides. Exemplary solid supports include silica-based (e.g., silicon oxide, SiO₂), titania-based (e.g., titanium oxide, TiO₂), germanium-based (e.g., germanium oxide), zirconia-based (e.g., zirconium oxide, ZrO₂), alumina-based (e.g., aluminum oxide, Al₂O₃) materials or mixtures thereof. Other solid supports include cross-linked and non-crosslinked polymers, carbonized materials and metals.

The solid support may be formed from any synthetic resin material. Exemplary synthetic polymer ion-exchange resins include poly(phenol-formaldehyde), poly(acrylic acid), poly(methacrylic acid), polynitriles, amine-epichlorohydrin copolymers, graft polymers of styrene on polyethylene or polypropylene, poly(2-chloromethyl-1,3-butadiene), poly(vinylaromatic) resins such as those derived from styrene, alpha-methylstyrene, chlorostyrene, chloromethylstyrene, vinyltoluene, vinylnaphthalene or vinylpyridine, corresponding esters of acrylic acid and methacrylic acid, and similar unsaturated monomers, mono-vinylidene monomers including the monovinylidine ring-containing nitrogen heterocyclic compounds, and any copolymers of the above resins.

Any of the above materials can optionally be co-polymerized with monomers incorporating ionic or ionizable functionalities. Any of the above materials can optionally be functionalized with a suitable ligand incorporating ionic or ionizable functionalities.

In one embodiment, the solid support comprises cross-linked polymers or copolymers. An exemplary copolymer is styrene-divinylbenzene copolymer (e.g., PS-DVB). In one example, the styrene-divinylbenzene copolymer contains between about 0% to about 100% divinylbenzene monomer by weight. In another example, the styrene-divinylbenzene copolymer contains between about 25% to about 80% divinylbenzene monomer by weight. The copolymer can be prepared, for example, according to the method of Ikada et al., Journal of Polymer Science, Vol. 12, 1829-1839 (1974) or as described in U.S. Pat. No. 4,382,124 to Meitzner, et al.

In one embodiment, the solid support is a silica-based substrate. Exemplary silica-based solid supports include silica gel, glass, sol-gels, polymer/sol-gel hybrids and silica monolithic materials.

The solid support can be of any form, including particulates (e.g., spherical, essentially spherical; e.g., resin beads), chips, chunks, blocks, monoliths and the like. When the solid support is in particulate form, the particles (e.g., irregular-shaped or bead-shaped, e.g., essentially spherical) have a median particle size (i.e., diameter). In one example, the median particle size of the solid support (e.g., spherical silica gel) is between about 0.1 (e.g., silica micro-spheres) and about 10,000 μm (microns). In one example, the median particle size of the solid support is between about 1 and about 5000 microns, between about 1 and about 1000 microns, between about 1 and about 500 microns, between about 1 and about 400 microns, between about 1 and about 300 microns, between about 1 and about 200 microns or between about 1 and about 100 microns. In yet another example, the median particle size of the solid support is between about 1 and about 80 microns, between about 1 and about 70 microns, between about 1 and about 60 microns, between about 1 and about 50 microns, between about 1 and about 40 microns, between about 1 and about 30 microns, between about 1 and about 20 microns or between about 1 and about 10 microns. In other example, the median particle size of the solid support particles is between about 10 and about 100 microns, between about 10 and about 80 microns, between about 40 and about 200 microns, between about 40 and about 100 microns, between about 40 and about 80 microns, between about 60 and about 200 microns, between about 60 and about 100 microns, between about 70 and about 200 microns, between about 80 and about 200 microns, between about 100 and about 200 microns, between about 200 and about 600 microns, between about 200 and about 500 microns or between about 200 and about 400 microns. In a particular example, the solid support is silica-based (e.g., silica gel) having a median particle size of between about 10 and 150 microns. The particle size can also be measured in “mesh” as defined on the Tyler Equivalent scale (the smaller the particle, the higher the mesh number). Typical mesh characteristics range between about 10 and 600. Generally, solid support particles useful in any packed bed chromatographic application (e.g., LC, HPLC or ultra-pressure chromatography) are suitable for use as the porous solid of the present disclosure.

In embodiments, the solid support is in particulate form, and multiple support particles are disposed in a packed bed. For example, a plastic or metal column is packed with the support particles.

In embodiments, the solid support particles are essentially “monodisperse” or essentially “homodisperse”, which indicates that the particle size of the majority of the particles (e.g., 80, 90 or 95% of the particles) does not vary substantially (e.g., not more than 50%) below or above the median particle size (M). In an exemplary monodisperse solid support particle population, 90% of the particles have an average particle size of between about 0.5×M and about 1.5×M.

The pores of the solid support particles can have any size. In a typical solid support, the average pore size is equal to or smaller than the micro-particles, described herein below. The nominal pore size is typically measured in angstroms (10-10 m, Å). In one example, the average diameter of the solid support pores is between about 1 and about 5000 Å. In another example, the volume average diameter of the solid support pores is between about 10 and about 5000 Å, between about 10 and about 4000 Å, between about 10 and about 3000 Å, between about 10 and about 2000 Å, between about 10 and about 1000 Å, between about 10 and about 800 Å, between about 10 and about 600 Å, between about 10 and about 400 Å, between about 10 and about 200 Å, between about 10 and about 100 Å, between about 20 and about 200 Å, between about 20 and about 100 Å, between about 30 and about 200 Å, between about 30 and about 100 Å, between about 40 and about 200 Å, between about 40 and about 100 Å, between about 50 and about 200 Å, between about 50 and about 100 Å, between about 60 and about 200 Å, between about 60 and about 100 Å, between about 70 and about 200 Å, between about 70 and about 100 Å, between about 80 and about 200 Å, between about 100 and about 200 Å, between about 100 and about 300 Å, between about 100 and about 400 Å, between about 100 and about 500 Å, between about 200 and about 500 Å, or between about 200 and about 600 Å.

The pores of the substrate can have any size. In a typical substrate, the average pore size is equal to or smaller than the micro-particles, described herein below. The nominal pore size is typically measured in angstroms (10-10 m, Å). In one example, the average diameter of the substrate pores is between about 1 and about 5000 Å. In another example, the volume average diameter of the substrate pores is between about 10 and about 5000 Å, between about 10 and about 4000 Å, between about 10 and about 3000 Å, between about 10 and about 2000 Å, between about 10 and about 1000 Å, between about 10 and about 800 Å, between about 10 and about 600 Å, between about 10 and about 400 Å, between about 10 and about 200 Å, between about 10 and about 100 Å, between about 20 and about 200 Å, between about 20 and about 100 Å, between about 30 and about 200 Å, between about 30 and about 100 Å, between about 40 and about 200 Å, between about 40 and about 100 Å, between about 50 and about 200 Å, between about 50 and about 100 Å, between about 60 and about 200 Å, between about 60 and about 100 Å, between about 70 and about 200 Å, between about 70 and about 100 Å, between about 80 and about 200 Å, between about 100 and about 200 Å, between about 100 and about 300 Å, between about 100 and about 400 Å, between about 100 and about 500 Å, between about 200 and about 500 Å or between about 200 and about 600 Å.

The specific surface area of the solid support is typically between about 0.1 and about 2,000 m²/g. For example, the specific surface area of the solid support is between about 1 and about 1,000 m²/g, between about 1 and about 800 m²/g, between about 1 and about 600 m²/g, between about 1 and about 400 m²/g, between about 1 and about 200 m²/g or between about 1 and about 100 m²/g of solid support. In another example, the specific surface area of the solid support is between about 3 and about 1,000 m²/g, between about 3 and about 800 m²/g, between about 3 and about 600 m²/g, between about 3 and about 400 m²/g, between about 3 and about 200 m²/g or between about 3 and about 100 m 2/g of solid support. In yet another example, the specific surface area of the solid support is between about 10 and about 1,000 m²/g, between about 10 and about 800 m²/g, between about 10 and about 600 m 2/g, between about 10 and about 400 m²/g, between about 10 and about 200 m²/g or between about 10 and about 100 m²/g of solid support.

In one embodiment, the solid support (e.g., silica gels or synthetic organic resins) have an exterior surface and pore openings defined by “interior walls” with an interior diameter defining the pore size. The pores open to the exterior surface of the solid support. The solid support includes ion exchange groups, which are positively charged groups. In one example, the ion-exchange groups are provided by the solid support itself, e.g., by incorporation of charged monomers into a synthetic resin polymer or by ionizable silanol groups on the surface of a silica substrate. In another example, the solid support (e.g., silica gel, silica monoliths) is covalently modified (e.g., alongside the interior pore walls and optionally the exterior surface) with organic ion-exchange ligands (e.g., silyl ligands). The ligands incorporate at least one ion-exchange group (e.g., ionic or ionizable group). The ionic nature of the ligand is positive.

Exemplary ion-exchange groups include anion-exchange groups, such as amino groups (e.g., secondary, tertiary or quaternary amines). Other anion-exchange groups are contemplated, such as phosphonium groups.

The porous solid of the present disclosure further comprises anions which balance the charge of the positively charged anion exchange groups chemically bonded to the solid support.

Exemplary anions include hydrogen carbonate, carbonate, hydrogen phosphate, phosphate, and chloride. Preferred anions include hydrogen carbonate and carbonate. Particularly preferred anions include hydrogen carbonate. Other suitable anions are contemplated.

Porous solids useful in the process of the present disclosure are commercially available in the form of cartridges. For example, a bicarbonate form cartridge, READI-CLING™, PS-HCO₃ SAX (Huayi Isotopes Co.), a carbonate form cartridge, Sep-Pak Light QMA Carbonate (Waters™) and a chloride form cartridge, Sep-Pak Accell Plus QMA Plus Light Cartridge (Waters™).

Process to Prepare [⁸⁹Zr]ZrCl₄ Solution

In an exemplary embodiment, [⁸⁹Zr][Zr(oxalate)₄]⁴⁻ in aqueous oxalic acid solution is contacted with the porous solid as herein disclosed. Subsequently, the porous solid is treated with an aqueous acidic solution comprising chloride ions, for example HCl solution, and a solution of [⁸⁹Zr]ZrCl₄ then recovered from the porous solid.

In a particular embodiment, the porous solid is in the form of a packed bed, for example packed within a cartridge. The [⁸⁹Zr][Zr(oxalate)₄]⁴⁻ in oxalic acid solution is introduced onto the packed bed and subsequently the packed bed is eluted with an aqueous acidic solution comprising chloride ions, for example HCl solution. The resulting eluent contains [⁸⁹Zr]ZrCl₄.

In embodiments, the concentration of HCl in the acidic solution comprising chloride ions is less than 1 M, or less than about 0.9 M, or less than about 0.8 M, or less than about 0.7 M, or less than about 0.6 M, or less than about 0.5 M, or less than about 0.4 M, or less than about 0.3 M, or less than about 0.2 M, or less than about 0.1 M.

In embodiments, the concentration of HCl in the acidic solution comprising chloride ions is from about 0.01 M to about 0.9 M, or from about 0.01 M to about 0.8 M, or from about 0.01 M to about 0.7 M, or from about 0.01 M to about 0.6 M, or from about 0.01 M to about 0.5 M, or from about 0.01 M to about 0.4 M, or from about 0.01 M to about 0.3 M, or from about 0.01 M to about 0.2 M, or from about 0.01 M to about 0.1 M.

In embodiments, the concentration of HCl in the acidic solution comprising chloride ions is from about 0.05 M to about 0.9 M, or from about 0.05 M to about 0.8 M, or from about 0.05 M to about 0.7 M, or from about 0.05 M to about 0.6 M, or from about 0.05 M to about 0.5 M, or from about 0.05 M to about 0.4 M, or from about 0.05 M to about 0.3 M, or from about 0.05 M to about 0.2 M, or from about 0.05 M to about 0.1 M.

In embodiments, the acidic solution comprising chloride ions comprises an alkali metal salt, for example sodium chloride.

In embodiments the concentration of alkali metal salt in the acidic solution comprising chloride ions is from about 0.1 M to about 2 M, or from about 0.5 M to about 1.5 M.

In embodiments, the process is free of organic solvents. In particular embodiments, the process is free of toxic organic solvents, for example the process is free of acetonitrile.

In embodiments, the solution comprising [⁸⁹Zr]ZrCl₄ has a pH which is greater than about 1, or greater than about 2, or greater than about 3, or greater than about 4, or greater than about 5, or greater than about 6.

In embodiments, the solution comprising [⁸⁹Zr]ZrCl₄ may be directly utilised in the preparation of radiopharmaceuticals, obviating the need for pH adjustment through addition of buffer which disadvantageously dilutes the concentration of 89-zirconium and further removes the requirement for additional purification steps.

### Process to Prepare Radiopharmaceuticals

In another aspect the present disclosure provides a process for the synthesis of a ⁸⁹Zr labelled radiopharmaceutical comprising the step of contacting a solution of [⁸⁹Zr]ZrCl₄ formed by the process according to any one of the herein disclosed embodiments with a biomarker targeting agent, said biomarker targeting agent comprising one or more moieties capable of forming a complex with zirconium of coordination number six to eight.

The biomarker targeting agent may comprises a small molecule, or a peptide. The small molecule may have a molecular weight of less than 1000 Dalton.

In embodiments, the biomarker targeting agent comprises one or more of polypeptide, protein, and antibody.

In embodiments, the one or more moieties capable of forming a complex with zirconium is a chelator.

In embodiments, the chelator comprises one or more nitrogen, oxygen, or sulphur atoms.

The skilled person would appreciate that a wide range of chelators may be utilised to prepare biomarker targeting agents suitable for use in the presently disclosed radiolabeling processes. A majority of useful chelators bear hydroxamate groups. Reference is made to Feiner et al., Cancers, 2021, 13, 4466, which is incorporated by reference in its entirety and which describes both hydroxamate chelators and other classes of chelators.

In embodiments, the chelator is selected from DFO-squaramide, DFO*-squaramide, benzyl isothiocyanate-DFO, benzyl isothiocyanate-DFO*, wherein DFO is desferrioxamine B and DFO* is desferrioxamine*, and DOTA.

In embodiments, the biomarker targeting agent is selected from DFOSq-bisPSMA, DFOSq-octreoTATE, DFOSq-girentuximab, and DOTA-octreotate.

### Process Automation

Embodiments of the present disclosure provide a process for synthesising radiolabelled pharmaceuticals wherein one or more steps of the process is automated.

In an embodiment, the automated process may be performed using a disposable cassette based MultiSyn radiosynthesiser (iPHASE Technologies Pty Ltd, Australia).

### EXAMPLES

### General Methods

READI-CLING PS-HCO3 strong anion exchange cartridge in hydrogen carbonate form was sourced from Huayi Isotope Co. Sep-Pak Light QMA Accell Plus and QMA-Carbonate Plus Light strong anion exchange cartridges in, respectively, chloride and carbonate forms were sourced from Waters, Australia.

1 M hydrochloric acid (GMP) and 1 M sodium chloride (GMP) were sourced from Merck. Sodium acetate (GMP) and gentisic acid (GMP) were sourced from Huayi Isotope Co.

Zirconium-89 was produced at Austin Health (Heidelberg, VIC) via the 89Y(p,n)⁸⁹Zr reaction using an IBA (Belgium) 18 MeV cyclotron and reconstituted in 0.05 M oxalic acid (Sigma Aldrich, USA, purified grade, 99.999% trace metal basis dissolved in Ultrapur water). Radioactivity was measured using either a Capintec CRC-55t PET dose calibrator (Mirion Technologies Inc., USA) or a Perkin Elmer (USA) Wizard2 automated gamma counter. DFOSq-bisPSMA (GMP) was sourced from Auspep, Australia.

### Example 1: Preparation of [⁸⁹Zr]ZrCl₄ Solution with PS-HCO₃ Cartridge

1.8 ml of 1 M sodium chloride solution was combined with 0.2 ml 1M HCl solution to provide 2 ml of a solution having a sodium chloride concentration of about 1 M and a HCl concentration of 0.1 M.

A solution of [⁸⁹Zr]Zr-oxalate in oxalic acid was loaded onto a bicarbonate activated ion exchange cartridge (READI-CLING PS-HCO₃, a strong basic anion exchange resin based on polystyrene-divinylbenzene in HCO₃ form) containing approximately 40 mg of the adsorbent.

The adsorbent was subsequently washed with 50 ml MiliQ water and then eluted with 0.5 ml of the HCl/NaCl solution to provide a solution of [⁸⁹Zr]ZrCl₄. Typically greater than 85% recovery of ⁸⁹Zr as the chloride resulted.

### Example 2: Preparation of [⁸⁹Zr]Zr-DFOSq-bisPSMA

1 mg of DFOSq-bisPSMA was dissolved in 1 ml of a 1:1 mixture of ethanol/water. To the mixture (50 μL) was added 60 μL of 3 M sodium acetate and 75 μl of 0.5% gentisic acid solution in water.

The solution of [⁸⁹Zr]ZrCl₄ from Example 1 was combined with the solution of DFOSq-bisPSMA and heated at 75° C. for 15 min to provide [⁸⁹Zr]Zr-DFOSq-bisPSMA.

### Example 3: Automated Synthesis of [⁸⁹Zr]Zr-DFOSq-bisPSMA

Utilising the reagents and protocols in Examples 1 and 2 the automated synthesis of [⁸⁹Zr]Zr-DFOSq-bisPSMA from [⁸⁹Zr]Zr-oxalate in oxalic acid and DFOSq-bisPSMA was performed using a MultiSyn radiosynthesiser (iPHASE Technologies Pty Ltd, Australia).

The % recovery of [⁸⁹Zr]ZrCl₄ was greater than 80%, and greater than 95% radiochemical yield of [⁸⁹Zr]Zr-DFOSq-bisPSMA resulted.

### Example 4: Automated Synthesis of [⁸⁹Zr]Zr-DFOSq-Octreotate

Following the protocol of Example 3, the automated synthesis of [⁸⁹Zr]Zr-DFOSq-octreotate from [⁸⁹Zr]Zr-oxalate in oxalic acid and DFOSq-octreotate was performed using a MultiSyn radiosynthesiser (iPHASE Technologies Pty Ltd, Australia).

The % recovery of [⁸⁹Zr]ZrCl₄ was greater than 80%, and greater than 97% radiochemical yield of [⁸⁹Zr]Zr-DFOSq-octreotate resulted.

### Example 5: Automated Synthesis of [⁸⁹Zr]Zr-DFOSq-Girentuximab

Following the protocol of Example 3, the automated synthesis of [⁸⁹Zr]Zr-DFOSq-girentuximab from [⁸⁹Zr]Zr-oxalate in oxalic acid and DFOSq-girentuximab was performed using a MultiSyn radiosynthesiser (iPHASE Technologies Pty Ltd, Australia).

The % recovery of [⁸⁹Zr]ZrCl₄ was greater than 80%, and greater than 97% radiochemical yield of [⁸⁹Zr]Zr-DFOSq-girentuximab resulted.

These automated syntheses results contrast with recently reported results (Wichmann, C. W., et al, Nuclear Medicine and Biology, 120-121 (2023) 108351) on the automated synthesis of [⁸⁹Zr]Zr-DFOSq-durvalumab from [⁸⁹Zr]Zr-oxalate, using a similar MultiSyn radiosynthesiser, in which radiochemical yields of only 75% were obtained.

A further advantage of the presently disclosed processes is that the typically utilised PD-10 column for oxalate removal (as taught in Wichmann et al) may be eliminated, highlighting the usefulness of [⁸⁹Zr]ZrCl₄ as a source of ⁸⁹Zr.

### Example 6: Further Preparations of [⁸⁹Zr]ZrCl₄ Solutions with PS-HCO₃ Cartridge

Example 1 was repeated except that the nature of the eluting solvent was varied in terms of HCl and NaCl concentrations. Table 1 collects details of the preparations and the results.

TABLE 1
[⁸⁹Zr]ZrCl₄ production and radiolabelling using PS-HCO₃ cartridge

| | [⁸⁹Zr]Zr- | | | | % RCY |
|---|---|---|---|---|---|
| | oxalate | Oxalic | | | with |
| | loaded | acid conc. | | % Recovery | DFOSq- |
| Run # | (MBq) | (M) | Eluting solvent | [⁸⁹Zr]ZrCl₄ | BisPSMA |
| 1 | 40 | 0.05 | 1M HCl | 90 | >95 |
| 2 | 40 | 0.05 | 5M NaCl/0.1M HCl | 88 | >95 |
| 3 | 44 | 0.05 | 2.5M NaCl/0.05M HCl | 93 | >95 |
| 4 | 45 | 0.05 | 2.5M NaCl/0.05M HCl | 87 | >95 |
| 5 | 100 | 0.05 | 2.5M NaCl/0.05M HCl | 92 | >95 |
| 6 | 110 | 0.05 | 1M NaCl/0.1M HCl | 88 | >95 |
| 7 | 100 | 0.05 | 1M NaCl/0.1M HCl | 88 | >95 |
| 8 | 64 | 0.5 | 2.5M NaCl/0.05M HCl | 92 | >95 |
| 9 | 40 | 1 | 2.5M NaCl/0.05M HCl | 93 | >95 |
| 10 | 48 | 0.05 | 0.1M HCl | 23 | Not tested |

It is noted that replacing the 1 M HCl (run #1) with a mixture of more dilute HCl and NaCl resulted in comparable [⁸⁹Zr]ZrCl₄ recovery amounts. Moreover, runs 6 and 7 illustrate that high percentage recoveries were also observed with high radiochemical loadings of around 100 MBq. The results are surprising and advantageous as they obviate the need to dilute the acidic [⁸⁹Zr]ZrCl₄ solution for further use.

In contrast, replacing 1 M HCl with 0.1M HCl, but with no added NaCl, afforded poorer recovery of [⁸⁹Zr]ZrCl₄. See run #10 in Table 1, indicating only 23% recovery. Additionally, eluting run #10 with a further 0.1 M HCl/1 M NaCl mixture, resulted in a total of 98% [⁸⁹Zr]ZrCl₄ recovery.

Utilising the [⁸⁹Zr]ZrCl₄ solutions prepared in runs 1-9 to prepare [⁸⁹Zr]Zr-DFOSq-bisPSMA as per Example 2 resulted in greater than 95% radiochemical yield (RCY) in each case.

### Example 7: Preparations of [⁸⁹Zr]ZrCl₄ Solutions with QMA-CI Cartridge

Example 1 was repeated except that a cartridge in chloride form was utilised and the nature of the eluting solvent was varied in terms of HCl and NaCl concentrations. Note that a chloride form cartridge requires, per manufacturer's instructions, an organic solvent, typically acetonitrile, to activate. Table 2 collects details of the preparations and the results.

TABLE 2
[⁸⁹Zr]ZrCl₄ production and radiolabelling using QMA-Chloride cartridge

| | | [⁸⁹Zr]Zr- | | | | % RCY |
|---|---|---|---|---|---|---|
| | | oxalate | | [⁸⁹Zr]ZrCl₄ | | with |
| | Activating | loaded | HCl conc. | recovery | % Recovery | DFOSq- |
| Run # | solvent | (MBq) | (M) | (MBq) | [⁸⁹Zr]ZrCl₄ | BisPSMA |
| 11 | acetonitrile | 52 | 1 | 43 | 83 | >90 |
| 12 | acetonitrile | 150 | 1 | 140 | 93 | >90 |
| 13 | ethanol | 64 | 1 | 35 | 55 | not tested |
| 14 | DMSO | 33 | 1 | 25 | 76 | ~50 |
| 15 | acetonitrile | 40 | 0.25 | 32 | 80 | ~40 |
| 16 | acetonitrile | 58 | 0.5 | 43 | 74 | >80 |
| 17 | acetonitrile | 50 | 1 | 47 | 94 | >95 |

It is noted that reducing the eluent HCl concentration to 0.25 M (run #15) resulted in only 80% [⁸⁹Zr]ZrCl₄ recovery and only ˜40% radiochemical yield with DFOSq-BisPSMA. The data in Table 2 illustrate that prior art protocols which use acetonitrile to activate a chloride cartridge require high concentration of acid (1 M) to achieve useful recoveries.

Activation of the cartridge with ethanol or DMSO in place of acetonitrile and elution with 1 M HCl solution resulted in poor recoveries.

### Example 8: Preparation of [⁸⁹Zr]ZrCl₄ Solution with QMA-Carbonate Cartridge

The procedure of Example 1 was repeated except that a QMA-carbonate form cartridge containing 130 mg sorbent was utilised. The cartridge was activated with 6 ml acetonitrile and loaded with a solution of [⁸⁹Zr]Zr-oxalate (55 MBq in 40 μl) in oxalic acid (0.05M). Elution with 1 mL of 0.1M HCl: 1M NaCl solution produced 38 MBq as [⁸⁹Zr]ZrCl₄ while approximately 10 MBq remained on the cartridge. The overall recovery was 69%.

1. A process for the synthesis of [⁸⁹Zr]ZrCl₄ solution comprising:
(a) contacting a solution comprising [⁸⁹Zr][Zr(oxalate)₄]⁴⁻ salt with a porous solid having anion exchange capacity, said porous solid comprising ligands covalently attached thereto, said ligands comprising ion exchange groups having a positive charge;
(b) treating the porous solid with an acidic solution comprising chloride ions; and
(c) recovering a solution comprising [⁸⁹Zr]ZrCl₄ from the porous solid.

2. The process according to claim 1, wherein the porous solid having anion exchange capacity comprises hydrogen carbonate ions, carbonate ions, hydrogen phosphate ions, phosphate ions, chloride ions, or mixtures thereof.

3. The process according to claim 2, wherein the porous solid having anion exchange capacity comprises hydrogen carbonate ions, carbonate ions, or mixtures thereof.

4. The process according to claim 2, wherein the porous solid having anion exchange capacity comprises hydrogen carbonate anions.

5. The process according to any one of claims 1 to 4, wherein the porous solid is in particulate form.

6. The process according to any one of claims 1 to 5, wherein the porous solid is disposed in a packed bed.

7. The process according to any one of claims 1 to 6, wherein the porous solid comprises synthetic organic polymer, silica or alumina.

8. The process according to claim 7, wherein the synthetic organic polymer comprises crosslinked polystyrene-divinylbenzene.

9. The process according to any one of claims 1 to 8, wherein the ligands comprising ion exchange groups having a positive charge comprise quaternary ammonium groups, or quaternary phosphonium groups.

10. The process according to any one of claims 1 to 9, wherein the acidic solution comprising chloride ions comprises HCl.

11. The process according to claim 10, wherein the concentration of HCl in the acidic solution comprising chloride ions is less than 1 M, or less than about 0.5 M, or less than about 0.2 M.

12. The process according to claim 10, wherein the concentration of HCl in the acidic solution comprising chloride ions is from about 0.01 M to less than 1 M, or from about 0.05 M to about 0.5 M, or from about 0.05 M to about 0.2 M.

13. The process according to claim 10, wherein the concentration of HCl in the acidic solution comprising chloride ions is from about 0.05 M to about 0.2 M.

14. The process according to any one of claims 1 to 13, wherein the acidic solution comprising chloride ions further comprises alkali metal chloride.

15. The process according to claim 14, wherein the concentration of alkali metal chloride is from about 0.1 M to about 2 M, or from about 0.5 M to about 1.5 M.

16. The process according to any one of claims 1 to 9, wherein the porous solid having anion exchange capacity comprises hydrogen carbonate ions, the acidic solution comprising chloride ions comprises HCl in a concentration less than 1 M, or less than about 0.5 M, or less than about 0.2 M, and the acidic solution comprising chloride ions further comprises alkali metal chloride, for example sodium chloride, and the concentration of alkali metal chloride is from about 0.1 M to about 2 M.

17. The process according to any one of claims 1 to 16, wherein the solution comprising [⁸⁹Zr]ZrCl₄ has a pH greater than 1.

18. The process according to any one of claims 1 to 17, wherein the process is free of organic solvents.

19. A process according to any one of claims 1 to 18, wherein the yield of [⁸⁹Zr]ZrCl₄ is at least about 80%, or at least about 85%, or at least about 90%, or at least about 95%, based on [⁸⁹Zr][Zr(oxalate)₄]⁴⁻ salt.

20. A process for the synthesis of a ⁸⁹Zr labelled radiopharmaceutical comprising the step of contacting the solution of [⁸⁹Zr]ZrCl₄ formed by the process according to any one of claims 1 to 19 with a biomarker targeting agent, said biomarker targeting agent comprising one or more moieties capable of forming a complex with zirconium of coordination number six to eight.

21. The process according to claim 20, wherein the biomarker targeting agent comprises a small molecule, or a peptide.

22. The process according to claim 21, wherein the small molecule has a molecular weight of less than 1000 Dalton.

23. The process according to claim 20, wherein the biomarker targeting agent comprises one or more of polypeptide, protein, and antibody.

24. The process according to any one of claims 20 to 23, wherein the one or more moieties capable of forming a complex with zirconium is a chelator.

25. The process according to claim 24, wherein the chelator comprises one or more nitrogen, oxygen, or sulphur atoms.

26. The process according to claim 24 or claim 25, wherein the chelator is selected from DFO-squaramide, DFO*-squaramide, benzyl isothiocyanate-DFO, benzyl isothiocyanate-DFO*, wherein DFO is desferrioxamine B and DFO* is desferrioxamine*, and DOTA.

27. The process according to claim 20, wherein the biomarker targeting agent is selected from DFOSq-bisPSMA, DFOSq-octreoTATE, DFOSq-girentuximab, and DOTA-octreotate.

28. The process according to any one of claims 1 to 27, wherein one or more of the process steps is automated.

29. A solution of [⁸⁹Zr]ZrCl₄ formed by the process according to any one of claim 1 to 19, or 28.

30. A ⁸⁹Zr labelled radiopharmaceutical formed by the process according to any one of claims 20 to 28.

31. A ⁸⁹Zr labelled radiopharmaceutical formed by the process according to any one of claims 20 to 28 for use in the treatment of cancer in a patient.

32. A method of treating cancer in a patient, the method comprising administering to the patient the ⁸⁹Zr labelled radiopharmaceutical formed by the process according to any one of claims 20 to 28.

33. A ⁸⁹Zr labelled radiopharmaceutical formed by the process according to any one of claims 20 to 28 for use in targeting a biomarker in vivo.

34. A method of targeting a biomarker in vivo, comprising administering to a subject the ⁸⁹Zr labelled radiopharmaceutical formed by the process according to any one of claims 20 to 28.

35. The use according to claim 33, or the method according to claim 34, wherein the biomarker is PSMA, bombesin, CAIX, FAP, or HER2.
